(19) United States
(12) Patent Application Publication
(10) Pub. No.: US 2006/0235044 A1
(43) Pub. Date: Oct. 19, 2006

(54) Azabicyclononene derivatives

(76) Inventors: Olivier Bezencon, Riehen (CH); Daniel Bur, Allschwil (CH); Walter Fischli, Allschwil (CH); Lubos Remen, Allschwil (CH); Sylvia Richard-Bildstein, Dietwiller (FR); Thomas Weller, Binningen (CH)

Correspondence Address: HOXIE & TSO LLP, 374 MILLBURN AVENUE, SUITE 300 E, MILLBURN, NJ, 07041, US

(21) Appl. No.: 10/554,879

(22) PCT Filed: Apr. 26, 2004
(86) PCT No.: PCT/EP04/04369

§ 371 Date: Oct. 28, 2005

(30) Foreign Application Priority Data
Apr. 30, 2003 (EP) EP03/04488

(51) Int. Cl. A61K 31/46; C07D 451/02

(52) U.S. Cl. 514/304; 546/124; 546/125

(57) ABSTRACT

The invention relates to novel 9-azabicyclo[3.3.1]nonene derivatives and related compounds and their use as active ingredients in the preparation of pharmaceutical compositions. The invention also concerns related aspects including processes for the preparation of the compounds, pharmaceutical compositions containing one or more of those compounds and especially their use as inhibitors of renin.

The invention relates to novel compounds of the general formula I. The invention also concerns related aspects including processes for the preparation of the compounds, pharmaceutical compositions containing one or more compounds of formula I and especially their use as renin inhibitors in cardiovascular events and renal insufficiency. Furthermore, these compounds can be regarded as inhibitors of other aspartyl proteases and might therefore be useful as inhibitors of plasmepsins to treat malaria and as inhibitors of Candida albicans secreted aspartyl proteases to treat fungal infections.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT₁ receptor (e.g. by losartan) on the other hand overexposes other AT-receptor subtypes to Ang II, whose concentration is dramatically increased by the blockade of AT₁ receptors. This may raise serious questions regarding the safety and efficacy profile of AT₁ receptor antagonists. In summary, renin inhibitors are not only expected to be different from ACE inhibitors and AT₁ blockers with regard to safety, but more importantly also with regard to their efficacy to block the RAS.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are missing and sought. Recently, the first non-peptide renin inhibitors were described which show high in vitro activity (Oefner C. et al., Chem. Biol., 1999, 6, 127; Patent Application WO97/09311; Märki H. P. et al., Il Farmaco, 2001, 56, 21). However, the development status of these compounds is not known.

The present invention relates to the identification of renin inhibitors of a non-peptidic nature and of low molecular weight. Orally active renin inhibitors of long duration of action which are active in indications beyond blood pressure regulation where the tissular renin-chymase system may be activated leading to pathophysiologically altered local functions such as renal, cardiac and vascular remodeling, atherosclerosis, and possibly restenosis are described.

The present invention describes non-peptidic renin inhibitors.

In particular, the present invention relates to novel compounds of the general formula I, 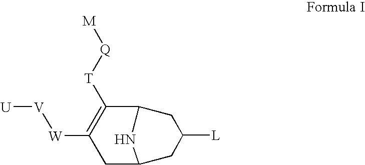
wherein
W is a six-membered, non benzofused, phenyl or heteroaryl ring, substituted by V in meta or para position;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; or —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents hydrogen; cycloalkyl; aryl; heterocyclyl; heteroaryl; aryl-O(CH₂)ᵥR⁵—; heteroaryl-O(CH₂)ᵥR⁵—; aryl-O(CH₂)₂O(CH₂)_{w}R⁵—; heteroaryl-(CH₂)₂O(CH₂)_{w}R⁵—;
L represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkinyl; cycloalkyl; aryl; cycloalkyl-lower alkyl;
R² and R²′ independently represent hydrogen; lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′, or lower alkyl, with the proviso that a carbon atom is attached at the most to one heteroatom in case this carbon atom is sp3-hybridized;
R⁴ and R⁴′ independently represent hydrogen; lower alkyl; cycloalkyl; cycloalkyl-lower alkyl; hydroxy-lower alkyl; —COOR²; —CONH₂;
R⁵ represents —OH, —OCOR², —COOR², —NR²R²′, —OCONR²R²′, —NCONR²R²′, cyano, —CONR²R²′, SO₃H, —SONR²R²′, —CO-morpholin-4-yl, —CO-((4-loweralkyl)piperazin-1-yl), —NH(NH)NH², —NR⁴R⁴′, with the proviso that a carbon atom is attached at the most to one heteroatom in case this carbon atom is sp3-hybridized;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3;
v is the integer 2, 3, or 4;
w is the integer 1 or 2;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

In the definitions of general formula I—if not otherwise stated—the term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl nad isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and consisting of two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and consisting of two to seven carbon atoms, preferably two to four carbon atoms, that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms, that can be optionally substituted by halogens. Examples of lower alkylene are ethylene, propylene or butylene.

The term lower alkenylene, alone or in combination with other groups, means straight and branched divalent chain groups comprising an olefinic bond and consisting of two to seven carbon atoms, preferably two to four carbon atoms, that can be optionally substituted by halogens. Examples of lower alkenylene are vinylene, propenylene and butenylene.

The term lower alkylenedioxy, refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono- or multisubstituted by lower alkyl, lower alkenyl, lower alkenylene, lower alkoxy, lower alkylenoxy, lower alkylenedioxy, hydroxy, halogen, —CF₃, —NR¹R¹′, —NR¹COR¹, —NR¹SO₂R¹′, —CONR¹R¹′, lower alkylcarbonyl, —COOR¹, —SR¹, —SOR¹, —SO₂R¹, —SO₂NR¹R¹′ whereby R¹¹ represents hydrogen; lower alkyl; lower alkenyl; lower alkinyl; cycloalkyl; aryl; cycloalkyl-lower alkyl. The cyclopropyl group is a preferred group.

The term aryl, alone or in combination, relates to the phenyl, the naphthyl or the indanyl group, preferably the phenyl group, which can be optionally mono- or multisubstituted by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R¹′, —NR¹R¹′-lower alkyl, —NR¹COR¹′, NR₁SO₂R¹′, —CONR¹R¹′, —NO₂, lower alkylcarbonyl, —COOR¹, —SR¹, —SOR¹, SO₂R¹, —SO₂NR¹R¹′, benzyloxy, whereby R¹′ has the meaning given above. Preferred substituents are halogen, lower alkoxy, lower alkyl, CF₃, OCF₃.

The term aryloxy refers to an Ar—O group, wherein Ar is an aryl. An example of a lower aryloxy group is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dibydropyrrolyl, imidazolidinyl, dihydropyrazolyl, pyrazolidinyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequatly substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R¹, —NR¹R¹′-lower alkyl, —N(R¹)COR¹, —N(R¹)SO₂R¹, —CONR¹R¹, —NO₂, lower alkylcarbonyl, —COOR¹, —SR¹, —SOR¹, —SO₂R¹, —SO₂NR¹R¹′, another aryl, another heteroaryl or another heterocyclyl and the like, whereby R¹′ has the meaning given above. Preferred heteroaryl are pyridinyl, pyrimidinyl, pyrazinyl, pyridazinyl.

The term heteroaryloxy refers to a Het-O group, wherein Het is a heteroaryl.

The term sp3-hybridized refers to a carbom atom and means that this carbon atom forms four bonds to four substituents placed in a tetragonal fashion around this carbon atom.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula I is acidic in nature with an inorganic base like an alkali or earth alkali base, e.g. sodium hydroxide, potassium hydroxide, calcium hydroxide and the like.

Compounds of the invention also include nitrosated compounds of the general formula I that have been nitrosated through one or more sites such as oxygen (hydroxyl condensation), sulfur (sulffiydryl condensation) and/or nitrogen. The nitrosated compounds of the present invention can be prepared using conventional methods known to one skilled in the art. For example, known methods for nitrosating compounds are described in U.S. Pat. Nos. 5,380,758 and 5,703,073; WO 97/27749; WO 98/19672; WO 98/21193; WO 99/00361 and Oae et al, Org. Prep. Proc. Int., 15(3): 165-198 (1983), the disclosures of each of which are incorporated by reference herein in their entirety.

The compounds of the general formula I can contain two or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

A group of preferred compounds of general formula I above are those wherein W, V, U, and L are as defined in general formula I and

T is —CONR¹—;

Q is methylene;

M is aryl; heteroaryl.

Another group of even more preferred compounds of general formula I are those wherein W, U, L, T, Q, and M are as defined in general formula I above and

V is —CH₂CH₂O—; —CH₂CH₂CH₂O—; —OCH₂CH₂O—.

Another group of also more preferred compounds of general formula I are those wherein V, U, T, Q, M, and L are as defined in general formula I above and

W represents a 1,4-disubstituted phenyl group.

Another group of also more preferred compounds of general formula I are those wherein W, V, U, T, Q, M, and L are as defined in general formula I above and

U is a mono-, di-, or trisubstituted phenyl or heteroaryl, wherein the substituents are halogen, lower alkyl, lower alkoxy, CF₃.

Especially preferred compounds of general formula I are those selected from the group consisting of:
(rac.)-(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-hydroxyethyl)amide],
(rac.)-(1R*,5S*,7R*)-3-{4-[3-(2-chloro-3,6-difluorophenoxy)propyl]phenyl}-9-azabicyclo[3.3.1]non-2-ene-2,7-dicarboxylic acid 7-benzylamide 2-[cyclopropyl-(3-methoxy-2-methylbenzyl)amide],
(rac.)-(1R*,5S*,7R*)-3-{4-[3-(2-chloro-3,6-difluorophenoxy)propyl]phenyl}-7-(4-methylpiperazine-1-carbonyl)-9-azabicyclo[3.3.1]non-2-ene-2-carboxylic acid cyclopropyl-(3-methoxy-2-methylbenzyl)amide,
(rac.)-(1R*,5S*,7R*)-3-{4-[3-(2-chloro-3,6-difluorophenoxy)propyl]phenyl}-9-azabicyclo[3.3. I]non-2-ene-2,7-dicarboxylic acid 7-cyclopropylamide 2-[cyclopropyl-(3-methoxy-2-methylbenzyl)amide],
(rac.)-(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-diethylamide,
(rac.)-(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-piperidin-1-ylethyl)amide],
(rac.)-(1R*,5S*,7R*)-3-{4-[3-(2-chloro-3,6-difluorophenoxy)propyl]phenyl}-7-(4-hydroxypiperidine-1-carbonyl)-9-azabicyclo[3.3.1]non-2-ene-2-carboxylic acid cyclopropyl-(3-methoxy-2-methylbenzyl)amide,
(rac.)-(1R*,5S*,7R*)-3-{4-[3-(2-chloro-3,6-difluorophenoxy)propyl]phenyl}-7-(2-hydroxymethylpyrrolidine-1-carbonyl)-9-azabicyclo[3.3.1]non-2-ene-2-carboxylic acid cyclopropyl-(3-methoxy-2-methylbenzyl)amide,
(rac.)-(1R*,5S*,7R*)-3-{4-[3-(2-chloro-3,6-difluorophenoxy)propyl]phenyl}-9-aza-bicyclo[3.3.1]non-2-ene-2,7-dicarboxylic acid 2-[cyclopropyl-(3-methoxy-2-methylbenzyl)amide]7-[(2-methoxyethyl)amide],
(rac.)-(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-methylamide,
({(rac.)-(1R*,3R*,5S*)-7-{4-[3-(2-chloro-3,6-difluorophenoxy)propyl]phenyl}-6-[cyclopropyl-(3-methoxy-2-methylbenzyl)carbamoyl]-9-azabicyclo[3.3.1]non-6-ene-3-carbonyl}amino)acetic acid methyl ester,
(rac.)-(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]non-6-ene-3-carboxylic acid,
(rac.)-(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]non-6-ene-3-carboxylic acid methyl ester,
(rac.)-(1R*,3R*,5S*)-3-{4-[3-(2-chloro-3,6-difluorophenoxy)propyl]phenyl}-7-methoxymethyl-9-azabicyclo[3.3.1]non-2-ene-2-carboxylic acid cyclopropyl-(3-methoxy-2-methylbenzyl)amide,
(rac.)-(1R*,3R*,5S*)-3-{4-[3-(2-chloro-3,6-difluorophenoxy)propyl]phenyl}-7-cyclopropoxymethyl-9-azabicyclo[3.3.1]non-2-ene-2-carboxylic acid cyclopropyl-(3-methoxy-2-methylbenzyl)amide,
(rac.)-(1R*,3R*,5S*)-7-aminomethyl-3-{4-[3-(2-chloro-3,6-difluorophenoxy)-propyl]phenyl}-9-azabicyclo[3.3.1]non-2-ene-2-carboxylic acid cyclopropyl-(3-methoxy-2-methylbenzyl)amide,
(rac.)-(1R*,3R*,5S*)-7-(acetylaminomethyl)-3-{4-[3-(2-chloro-3,6-difluoro-phenoxy)propyl]phenyl}-9-azabicyclo[3.3.1]non-2-ene-2-carboxylic acid cyclopropyl-(3-methoxy-2-methylbenzyl)amide,
(rac.)-(1R*,3R*,5S*)-3-{4-[3-(2-chloro-3,6-difluorophenoxy)propyl]phenyl}-7-dimethylaminomethyl-9-azabicyclo[3.3.1]non-2-ene-2-carboxylic acid cyclopropyl-(3-methoxy-2-methylbenzyl)amide, and
(rac.)-(1R*,3R*,5S*)-3-{4-[3-(2-chloro-3,6-difluorophenoxy)propyl]phenyl}-7-hydroxymethyl-9-azabicyclo[3.3.1]non-2-ene-2-carboxylic acid cyclopropyl-(3-methoxy-2-methylbenzyl)amide.

The compounds of general formula I and their pharmaceutically acceptable salts may be used as therapeutics e.g. in form of pharmaceutical compositions. These pharmaceutical compositions containing at least one compound of general formula I and usual carrier materials and adjuvants may especially be used in the treatment or prophylaxis of disorders which are associated with a dysregulation of the renin angiotensin system (RAS), comprising cardiovascular and renal diseases. Examples of such diseases are hypertension, congestive heart failure, pulmonary heart failure, coronary diseases, cardiac insufficiency, renal insufficiency, renal or myocardial ischemia, atherosclerosis, and renal failure. They can also be used to prevent restenosis after balloon or stent angioplasty, to treat erectile dysfunction, glomerulonephritis, renal colic, and glaucoma. Furthermore, they can be used in the therapy and the prophylaxis of diabetic complications, complications after vascular or cardiac surgery, complications of treatment with immunosuppresive agents after organ transplantation, complications of cyclosporin treatment, as well as other diseases presently known to be related to the RAS.

In another embodiment, the invention relates to a method for the treatment and/or prophylaxis of diseases which are related to the RAS such as hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppresive agents after organ transplantation, and other diseases which are related to the RAS, which method comprises administering a compound according of formula I to a human being or animal.

The invention further relates to the use of compounds of general formula I as defined above for the treatment and/or prophylaxis of diseases which are associated with the RAS such as hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppresive agents after organ transplantation, and other diseases presently known to be related to the RAS.

In addition, the invention relates to the use of compounds as defined above for the preparation of medicaments for the treatment and/or prophylaxis of diseases which are associated with the RAS such as hypertension, coronary diseases, cardiac insufficiency, renal insufficiency, renal and myocardial ischemia, and renal failure. These medicaments may be prepared in a manner known per se.

The compounds of formula I may also be used in combination with one or more pharmacologically active compounds e.g. with other renin inhibitors, with ACE-inhibitors, angiotensin II receptor antagonists, endothelin receptor antagonists, vasodilators, calcium antagonists, potassium activators, diuretics, sympatholitics, beta-adrenergic antagonists, alpha-adrenergic antagonists, and neutral endopeptidase inhibitors, for the treatment of disorders as above-mentioned.

All forms of prodrugs leading to an active component comprised by general formula I above are included in the present invention.

The compounds of general formula I can be manufactured by the methods outlined below, by the methods described in the examples or by analogous methods.

Chemistry

1,5-Dialdehydes can be prepared at best from cyclopentene derivatives (Scheme 1). Commercially available cyclopentene or cyclopent-3-enecarboxylic acid represent ideal starting materials. If necessary, the substituent R^{a} can be transformed in one or several steps into a substituent R^{b} suitable for the preparation of the final compounds (→compounds of type A). Oxydation to dialdehydes of type B may be conducted in two steps with OsO₄/NMO, then NaIO₄, or in one step with ozone.

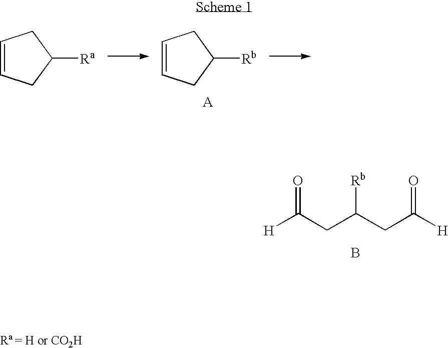

A double intramolecular Mannich condensation with at best methyl amine and 3-oxopentanedioic acid, and an aldehyde of type B, followed by a double decarboxylation, leads to an azabicyclononene of type C (Scheme 2). The R^{b}-substituent can exist both in an equatorial or in an axial position.

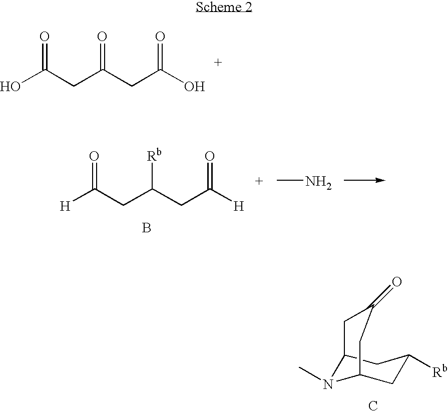

Acylation of bicyclononane C can occur racemically or enantioselectively as described in patent application WO03/093267 (Scheme 3). Bicyclononene of type D, whereas R^{c} is typically a methyl, an ethyl or a benzyl, can be obtained.

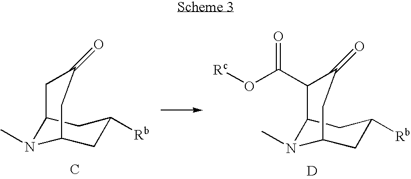

Then a similar chemistry may be used as described in an earlier patent application, and in patent applications WO03/093267 and WO04/002957. For instance bicyclononane D can be converted into the corresponding vinyl triflate E (Scheme 4). A suitable coupling with carbon-carbon bond formation (Suzuki, Negishi, Stille-couplings or similar ones) can lead to a bicyclononene derivative of type F, then protecting group manipulations can lead to a bicyclononene derivative of type G. R^{d} optionally represents any chemical precursor of a U-V group as defined in general formula I. Selective cleavage of an ester can lead to a bicyclononene derivative of type H, then an amide coupling to a bicyclononene derivative of type J. Standard manipulations at the R^{d}-substituent, like a Mitsunobu reaction can lead to a bicyclononene derivative of type K. If R^{b} is an ester, it can be hydrolyzed to a bicyclononene derivative of type N before a desired substituent L¹ being introduce (→compound of type M). L₁ can be then transformed into a substituent of type L as defined in general formula I. Finally, removal of the protecting group PG can lead to the desired final compound.

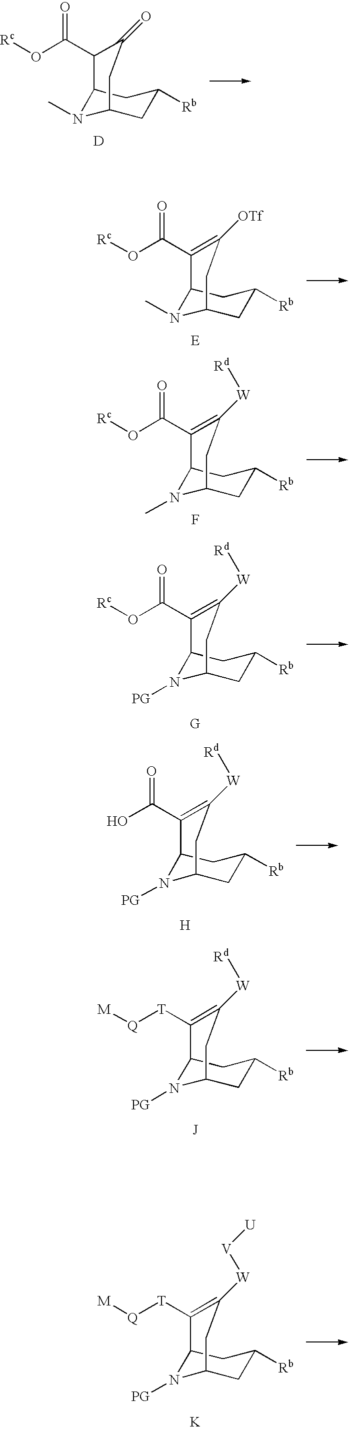

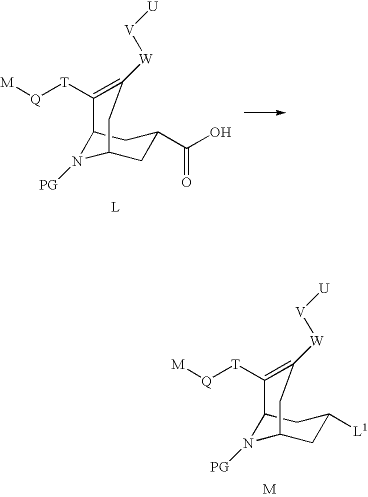

The compounds of formula I and their pharmaceutically acceptable acid addition salts can be used as medicaments, e.g. in the form of pharmaceutical preparations for enteral, parenteral, or topical administration. They can be administered, for example, perorally, e.g. in the form of tablets, coated tablets, dragées, hard and to soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula I and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of compounds of formula I can vary within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 50 mg to about 500 mg, comes into consideration.

The pharmaceutical preparations conveniently contain about 1-500 mg, preferably 5-200 mg of a compound of formula I.

The following examples serve to illustrate the present invention in more detail.

They are, however, not intended to limit its scope in any manner.

### EXAMPLES

Abbreviations

ACE Angiotensin Converting Enzyme

Ang Angiotensin

aq. aqueous

Bn Benzyl

Boc tert-Butyloxycarbonyl

BSA Bovine serum albumine

BuLi n-Butyllithium

conc. concentrated

DIPEA Diisopropylethylamine

DMAP 4-N,N-Dimethylaminopyridine

DMSO Dimethylsulfoxide

EDC HCl Ethyl-N,N-dimethylaminopropylcarbodiimide hydrochloride

EIA Enzyme immunoassay

eq. equivalent

Et Ethyl

EtOAc Ethyl acetate

FC Flash Chromatography

HOBt Hydroxybenzotriazol

KHMDS Potassium hexamethyldisilazide

MeOH Methanol

NMO N-Methylmorpholine N-oxide

org. organic

PG protecting group

Ph Phenyl

RAS Renin Angiotensin System

rt room temperature

sol. Solution

TBAF Tetra-n-butylanimonium fluoride

TBDMS tert-Butyldimethylsilyl

tBuOH tert-Butanol

tBuOK Potassium tert-butylate

Tf Trifluoromethylsulfonyl

THF Tetrahydrofuran

TLC Thin Layer Chromatography

Preparation of the Precursors

### 4-Oxo-2-(2-oxoethyl)butyric acid methyl ester (B)

To a sol. of cyclopent-3-enecarboxylic acid methyl ester (Lizotte, K. E.; et. al; J. Org. Chem., 1983, 48, 3594, 53 g, 0.420 mol) in MeOH (180 mL) was added water (270 mL). The mixture was cooled to −10° C. and O₃/O₂ was bubbled through for 5 h, while the temperature was maintained at −10° C. The mixture was stirred overnight under argon, while the temperature was allowed to raise to rt. A mixture of 3,3-thiodipropionic acid (100 g, 0.560 mol) dissolved in 5M NaOH (210 mL) and 2M NaOH (35 mL, final pH=7-8) was added under efficient stirring. The mixture was stirred for 30 min, and the solvents were partially removed under reduced pressure. The residue was saturated with NaCl and extracted with Et₂O (3×). The combined org. extracts were dried over Na₂SO₄, and filtered. Removing the solvents under reduced pressure yielded the title compound (42 g, 0.266 mol) as raw product that was directly engaged in the next step.

### (7r)-9-methyl-7-oxo-9-azabicyclo[3.3.1]nonane-3-carboxylic acid methyl ester (C)

A mixture of dialdehyde B (45.5 g, 0.288 mol) in water (1.755 L) was heated to the boiling point. An emulsion formed. The mixture was allowed to cool, and conc. aq. HCl (29.7 mL) was added. The mixture was cooled to rt and kept aside. Conc. aq. HCl (71.1 mL), then NaOH (23 g) were added to water (5185 mL). NaOAc (222.75 g, 2.72 mol) was added. Acetone dicarboxylic acid (103.2 g, 0.671 mol) was added. Methylamine hydrochloride (59.5 g, 0.864 mol) was added. The pH was measured at 6-7. To this mixture the aldehyde mixture prepared earlier was added dropwise over 15 min. The pH was measured at 4-4.5. The mixture was stirred for 24 h. NaHCO₃ was added until the mixture was clearly basic, and the mixture was saturated with Na₂SO₄. The mixture was extracted was tert-butylethylether (2×) and with butanol (2×). The ether extracts, and separately the butanol extracts were dried over Na₂SO₄, filtered, and the solvents were removed under reduced pressure. Purification by FC (toluene with 1% Et₃N, then EtOH) yielded the title compound (7 g, 12%).

The (7s)-isomer may have been present as minor isomer and could not be separated. Only the major (7r)-isomer will be considered hereby.

### (rac.)-(1R*,5S*,7R*)-9-Methyl-3-oxo-9-azabicyclo[3.3.1]nonane-2,7-dicarboxylic acid 2-benzyl ester 7-methyl ester (D)

A sol. of LDA was prepared from diisopropylamine (2.53 mL, 25 mmol), BuLi (1.6 M in hexanes, 15 mL, 24 mmol) and THF (75 mL). This sol. was cooled to −78° C. and a sol. of bicyclononane C (4.64 g, 22 mmol) in THF (10 mL) was added dropwise over 3 min. The reaction mixture was stirred for 1 h at −78° C., then benzylcyanoformat (4.86 g, 30 mmol) was added. The reaction mixture was stirred for 30 min. at −78° C. The reaction mixture was quenched with acetic acid (5 g, 83 mmol), allowed to warm to rt, and was partitioned between half-sat. brine (200 mL, pH 5-6) and chloroform (200 mL). The aq. phase was re-extracted with chloroform (100 mL), the combined organic phases were dried over MgSO₄, filtered, and the solvents were removed under reduced pressure. Purification of the residue by FC (MeOH/CHCl₃ 1:30→1:25) yielded the title compound (4.22 g, 56%) as an oil.

### (rac.)-(1R*,5S*,7R*)-9-Methyl-3-trifluoromethanesulfonyloxy-9-azabicyclo[33.1]non-2-ene-2,7-dicarboxylic acid 2-benzyl ester 7-methyl ester (E)

A sol. of bicyclononanone D (4.20 g, 12.2 mmol) in THF (65 mL) was cooled to 0° C. and NaH (about 60% in mineral oil, 0.70 g, about 16 mmol) was added. A gas evolution was observed. After 20 min, Tf₂NPh (6.35 g, 18 mmol) was added. 10 min later, the ice bath was removed. The sol. was stirred overnight, and diluted with EtOAc and washed with brine (1×). The org. extracts were dried over MgSO₄, filtered, and the solvents were removed under reduced pressure. Purification by FC yielded the title compound as an oil (4.11 g, 71%).

### (rac.)-(1R*,5S*,7R*)-3-{4-[3-(tert-Butyldimethylsilanyloxy)propyl]phenyl}-9-methyl-9-azabicyclo[3.3.1]non-2-ene-2,7-dicarboxylic acid 2-benzyl ester 7-methyl ester (F)

A sol. of [3-(4-bromophenyl)propoxy]-tert-butyldimethylsilane (Kiesewetter D. O., Tetrahedron Asymmetry, 1993, 4, 2183, 9.87 g, 30 mmol) in THF (150 mL) was cooled to −78° C. BuLi (1.6M in hexane, 18.8 mL, 30 mmol) was added. After 30 min, ZnCl₂ (1M in THF, 30 mL, 30 mmol, prepared from ZnCl₂ dried overnight at 150° C. and THF) was added. The mixture was allowed to warm up to rt. Vinyl triflate E (4.05 g, 8.48 mmol) in THF (30 mL) and then Pd(PPh₃)₄ (210 mg, 0.182 mmol) were added. The mixture was heated tro reflux for 90 min and aq. 1M HCl (1 mL) was added. The mixture was diluted with EtOAc and washed with aq. 1M NaOH (1×). The org. extracts were dried over MgSO₄, filtered and the solvents were removed under reduced pressure. Purification of the residue by FC yielded the title product (4.54 g, 92%).

### (rac.)-(1R*,5S*,7R*)-3-[4-(3-Hydroxypropyl)phenyl]-9-azabicyclo[3.3.1]non-2-ene-2,7,9-tricarboxylic acid 2-benzyl ester 9-tert-butyl ester 7-methyl ester (G)

1-Chloroethyl chloroformate (4.54 g, 32 mmol) was added to a sol. of bicyclononene F (4.44 g, 7.7 mmol) in 1,2-dichloroethane (60 mL). The sol. was heated to reflux. After 1 h, the reaction mixture was allowed to cool to rt, and the solvents were removed under reduced pressure. MeOH (50 mL) was added. The mixture was stirred at rt for 4 h, and the solvents were removed under reduced pressure. The residue was dissoled in CH₂Cl₂ (30 mL), DIPEA (2.0 g, 15.5 mmol) was added, and the mixture was cooled to 0° C. Boc₂O (1.97 g, 9.0 mmol) was added and the mixture was stirred at rt for 2 h. The mixture was washed with aq. 1M HCl (1×), and aq. sat. NaHCO₃ (1×). The org. extracts were dried over MgSO₄, filtered, and the solvents were removed under reduced pressure. Purification of the residue by FC yielded the title compound (2.29 g, 54%).

### (rac.)-(1R*,5S*,7R*)-3-{4-[3-(tert-Butyldimethylsilanyloxy)propyl]phenyl}-9-azabicyclo[3.3.1]non-2-ene-2,7,9-tricarboxylic acid 9-tert-butyl ester 7-methyl ester (H)

A mixture of compound G (2.07 g, 3.76 mmol) and Pd/C (10%, 300 mg) in EtOAc (50 mL) was hydrogenated at rt and atmospheric pressure. Hydrogen uptake ceased after the consumption of 1 equivalent hydrogen. The mixture was filtered through a bed of Celite and the solvents were removed under reduced pressure to leave an oil (1.71 g, 99%). This oil (1.37 g, 9.5 mmol), TBDMS-Cl (1.00 g, 14.7 mmol) and imidazole were dissolved in CH₂Cl₂ (25 mL) and the solution stirred at rt for 6 h (TLC-control). Aq. 5% NH4Cl (50 mL) was added and the mixure extracted with hexane (3×). The combined org. phases were dried over MgSO₄, filtered, and the solvents were removed under reduced pressure. The residual viscous oil (2.67 g) was dissolved in THF (35 mL), water (10 mL), and methanol (10 mL). K₂CO₃ (300 mg) was added and the clear solution stirred at rt for 1 h. 20% aq. NH₄Cl (50 mL) was added and the mixture extracted with tert-butylethylether (2×). The combined org. phases were dried over MgSO₄, filtered, and the solvents were removed under reduced pressure. The title compound (2.18 g, quantitative) was used without further purification.

### (rac.)-(1R*,3R*,5S*)-7-{4-[3-(tert-Butyldimethylsilanyloxy)-propyl]phenyl}-6-[cyclopropyl-(3-methoxy-2-methylbenzyl)carbamoyl]-9-azabicyclo[3.3.1]-non-6-ene-3,9-dicarboxylic acid 9-tert-butyl ester 3-methyl ester (J1)

A mixture of bicyclononene H (2.14 g, 3.73 mmol), cyclopropyl-(2-methyl-3-methoxybenzyl)amine (prepared by reductive amination from 3-methoxy-2-methylbenzaldehyde, Comins, D. L.; Brown, J. D., J. Org. Chem., 1989, 54, 3730, and cyclopropylamine; 2.45 g, 12.8 mmol), DIPEA (2.59 mL, 20 mmol), DMAP (175 mg, 1.4 mmol), HOBt (330 mg, 3.9 mmol) and EDC.HCl (2.88 g, 15 mmol) in CH₂Cl₂ (35 mL) was stirred at rt for 3 days. The mixture was diluted with more CH₂Cl₂, and washed with aq. 1M HCl (3×) and aq. sat. NaHCO₃ (1×). The org. extracts were dried over MgSO₄, filtered, and the solvents were removed under reduced pressure. Purification of the residue by FC yielded the title compound (2.09 g, 75%).

### (rac.)-(1R*,3R*,5S*)-6-[Cyclopropyl-(3-methoxy-2-methylbenzyl)-carbamoyl]-7-[4-(3-hydroxypropyl)phenyl]-9-azabicyclo[3.3.1]non-6-ene-3,9-dicarboxylic acid 9-tert-butyl ester 3-methyl ester (J2)

A solution of bicyclononene J1 (2.03 g, 2.7 mmol) in THF (30 mL) was cooled in an icebath. TBAF (1M in THF, 6 mL, 6 mmol) was added and the sol. stirred at 0° C. for 15 min and at rt for 1 h. The mixture was diluted with tert-butylmethylether (100 mL), washed with half-sat. brine (50 mL) and sat. brine (50 mL), dried over MgSO₄, filtered, and the solvents were removed under reduced pressure. The residual viscous oil was purified by FC (EtOAc/hexane 2:1) to yield the title compound (1.46 g, 86%).

### (rac.)-(1R*,3R*,5S*)-7-{4-[3-(2-Chloro-3,6-difluorophenoxy)propyl]-phenyl}-6-[cyclopropyl-(3-methoxy-2-methylbenzyl)-carbamoyl]-9-aza-bicyclo[3.3.1]non-6-ene-3,9-dicarboxylic acid 9-tert-butyl ester 3-methyl ester (K)

Tributylphosphine (1.73 mL, 7.7 mmol) was added to a sol. of bicyclononene J2 (1.44 g, 2.24 mmol), 2-chloro-3,6-difluorophenol (702 mg, 4.3 mmol) and azodicarboxylic dipiperidide (1.16 g, 4.6 mmol) in toluene (25 mL). The mixture was heated to reflux for 2 h and allowed to cool to rt. The solvents were removed under reduced pressure. Purification by FC yielded the title compound (1.68 g, 95%).

### (rac.)-(1R*,3R*,5S*)-7-{4-[3-(2-Chloro-3,6-difluorophenoxy)propyl]-phenyl}-6-[cyclopropyl-(3-methoxy-2-methylbenzyl)carbamoyl]-9-azabicyclo-[3.3.1]non-6-ene-3,9-dicarboxylic acid 9-tert-butyl ester (L)

A mixture of bicyclononene K (1.68, 7.2 mmol) in aq. 1M NaOH (25 mL) and MOH (25 mL) was stirred for 5 h at rt. The mixture was allowed to cool to rt and the solvents were partially removed under reduced pressure. The residue was acidified to pH 2 with aq. 1M HCl and this mixture was extracted with EtOAc (3×). The combined org. extracts were dried over MgSO₄, filtered, and the solvents were removed under reduced pressure. The crude title compound was used further without purification.

### (rac.)-(1R*,3R*,5S*)-3-{4-[3-(2-Chloro-3,6-difluorophenoxy)propyl]-phenyl}-2-[cyclopropyl-(3-methoxy-2-methylbenzyl)carbamoyl]-7-hydroxymethyl-9-azabicyclo[3.3.1]non-2-ene-9-carboxylic acid tert-butyl ester (M1)

A mixture of compound L (444 mg, 0.57 mmol) and LiBH4 (14.9 mg, 0.684 mmol) in EtOH (5 mL) was stirred at rt overnight. The mixture was diluted with Et₂O, and washed with water. The org. extracts were dried over Na₂SO₄, filtered, and the solvents were removed under reduced pressure. Purifcation of the crude by FC (EtOAc/heptane 1:1) yielded the title compound (327 mg, 76%). LC-MS: Rₜ=1.21 min; ES+: 751.25.

### (rac.)-(1R*,3R*,5S*)-3-{4-[3-(2-Chloro-3,6-difluorophenoxy)propyl]-phenyl}-2-[cyclopropyl-(3-methoxy-2-methylbenzyl)-carbamoyl]-7-(1,3-dioxo-1,3-dihydroisoindol-2-ylmethyl)-9-azabicyclo[3.3.1]non-2-ene-9-carboxylic acid tert-butyl ester (M2)

To a mixture of compound M1 (203 mg, 0.27 mmol) in toluene (1 mL) were added phthalimide (47.7 mg, 0.324 mmol), diethyl azodicarboxylic acid (62.7 μL, 0.405 mmol), and PPh₃ (142 mg, 0.54 mmol). The mixture was stirred at rt overnight. The reaction mixture was poured over diatomace earth (Isolute Sorbent Technology, Johnson, C. R., et al., Tetrahedron, 1998, 54, 4097; 0.5 g), and was treated with aq. 1M HCl (0.6 mL). The diatomace earth-suspension was left for 5 min, and was washed with CH₂Cl₂ (2×). The org. extracts were evaporated under reduced pressure. The residue was used without further purification. LC-MS: Rₜ=1.27 min; ES+: 880.15.

### (rac.)-(1R*,3R*,5S*)-7-Aminomethyl-3-{4-[3-(2-chloro-3,6-difluoro-phenoxy)propyl]phenyl}-2-[cyclopropyl-(3-methoxy-2-methylbenzyl)carbamoyl]-9-azabicyclo[3.3.1]non-2-ene-9-carboxylic acid tert-butyl ester (M3)

A mixture of compound M2 (238 mg, 0.27 mmol), aq. methyl amine (41%, 2 mL), and THF (2 mL) was stirred at rt for 1 h. The mixture was diluted with CH₂Cl₂, and washed with water. The org. extracts were dried over MgSO₄, filtered, and the solvents were removed under reduced pressure. The crude title compound was obtained as a yellow oil (323 mg), which was used further without purification.

### EXAMPLES

General Procedure A for Amide Coupling

A sol. of the desired carboxylic acid (1.00 eq), the desired amine (3.00 eq), EDC-HCl (1.50 eq.), HOBt (1.25 eq.), DMAP (cat. amount) and DIPEA (4.00 eq.) in CH₂Cl₂ (20 mL/g of acid) was stirred at rt overnight. The reaction mixture was poured over diatomace earth (Isolute Sorbent Technology, Johnson, C. R., et al., Tetrahedron, 1998, 54, 4097), treated with aq. 1M HCl, and the org. extracts were evaporated under reduced pressure. The residue was used without further purification.

General Procedure B for the Removal of a Boc-Protecting Group

The starting material was dissolved in CH₂Cl₂ (10 mL/g of starting material) and the sol. was cooled to 0° C. 4M HCl in dioxane (same volume as CH₂Cl₂) was added and the reaction mixture was left for 2 h at rt. The solvents were removed under reduced pressure. Purification of the residue by HPLC led to the desired compound.

### Example 1

### (rac.)-(1R*,5S*,7R*)-3-{4-[3-(2-Chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-hydroxyethyl)amide]

According to general procedures A and B, from bicyclononene L (0.05 mmol), and 2-aminoethanol. LC-MS: 0.91 min, MH+=708.25.

### Example 2

### (rac.)-(1R*,5S*,7R*)-3-{4-[3-(2-Chloro-3,6-difluorophenoxy)propyl]phenyl}-9-azabicyclo[3.3.1]non-2-ene-2,7-dicarboxylic acid 7-benzylamide 2-[cyclopropyl-(3-methoxy-2-methylbenzyl)amide]

According to general procedures A and B, from bicyclononene L (0.05 mmol), and benzylamine. LC-MS: 1.00 min, MH+=754.26.

### Example 3

### (rac.)-(1R*,5S*,7R*)-3-{4-[3-(2-Chloro-3,6-difluorophenoxy)propyl]phenyl}-7-(4-methylpiperazine-1-carbonyl)-9-azabicyclo[3.3.1]non-2-ene-2-carboxylic acid cyclopropyl-(3-methoxy-2-methylbenzyl)amide

According to general procedures A and B, from bicyclononene L (0.05 mmol), and N-methylpiperazine. LC-MS: 0.83 min, MH+=747.29.

### Example 4

### (rac.)-(1R*,5S*,7R*)-3-{4-[3-(2-Chloro-3,6-difluorophenoxy)propyl]phenyl}-9-azabicyclo[3.3.1]non-2-ene-2,7-dicarboxylic acid 7-cyclopropylamide 2-[cyclopropyl-(3-methoxy-2-methylbenzyl)amide]

According to general procedures A and B, from bicyclononene L (0.05 mmol), and cyclopropylamine. LC-MS: 0.96 min, MH+=704.27.

### Example 5

### (rac.)-(1R*,5S*,7R*)-3-{4-[3-(2-Chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-diethylamide

According to general procedures A and B, from bicyclononene L (0.05 mmol), and diethylamine. LC-MS: 0.99 min, MH+=720.27.

### Example 6

### (rac.)-(1R*,5S*,7R*)-3-{4-[3-(2-Chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-piperidin-1-ylethyl)amide]

According to general procedures A and B, from bicyclononene L (0.05 mmol), and 2-piperidin-1-ylethylamine. LC-MS: 0.85 min, MH+=775.29.

### Example 7

### (rac.)-(1R*,5S*,7R*)-3-{4-[3-(2-Chloro-3,6-difluorophenoxy)propyl]phenyl}-7-(4-hydroxypiperidine-1-carbonyl)-9-azabicyclo[3.3.1]non-2-ene-2-carboxylic acid cyclopropyl-(3-methoxy-2-methylbenzyl)amide

According to general procedures A and B, from bicyclononene L (0.05 mmol), and piperidin-4-ol. LC-MS: 0.92 min, MH+=748.29.

### Example 8

### (rac.)-(1R*,5S*,7R*)-3-{4-[3-(2-Chloro-3,6-difluorophenoxy)propyl]phenyl}-7-(2-hydroxymethylpyrrolidine-1-carbonyl)-9-azabicyclo[3.3.1]non-2-ene-2-carboxylic acid cyclopropyl-(3-methoxy-2-methylbenzyl)amide

According to general procedures A and B, from bicyclononene L (0.05 mmol), and pyrrolidin-2-ylmethanol. LC-MS: 0.85 min, MH+=748.28.

### Example 9

### (rac.)-(1R*,5S*,7R*)-3-{4-[3-(2-Chloro-3,6-difuorophenoxy)propyl]phenyl}-9-aza-bicyclo[3.3.1]non-2-ene-2,7-dicarboxylic acid 2-[cyclopropyl-(3-methoxy-2-methylbenzyl)amide]7-[(2-methoxyethyl)amide]

According to general procedures A and B, from bicyclononene L (0.05 mmol), and 2-methoxyethylamine. LC-MS: 0.94 min, MH+=722.26.

### Example 10

### (rac.)-(1R*,5S*,7R*)-3-{4-[3-(2-Chloro-3,6-difluorophenoxy)propyl]phenyl}-9-aza-bicyclo[3.3.1]non-2-ene-2,7-dicarboxylic acid 2-[cyclopropyl-(3-methoxy-2-methylbenzyl)amide]7-methylamide

According to general procedures A and B, from bicyclononene L (0.05 mmol), and methyl amine hydrochloride. LC-MS: 0.94 min, MH+=678.3.

### Example 11

### ({(rac.)-(1R*,5S*)-7-{4-[3-(2-chloro-3,6-difluorophenoxy)propyl]phenyl}-6-[cyclopropyl-(3-methoxy-2-methylbenzyl)carbamoyl]-9-azabicyclo[3.3.1]non-6-ene-3-carbonyl}amino)acetic acid methyl ester

According to general procedures A and B, from bicyclononene L (0.05 mmol), and glycine methyl ester hydrochloride. LC-MS: 0.95 min, MH+=736.25.

### Example 12

### (rac.)-(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]-non-6-ene-3-carboxylic acid

According to general procedure B, from bicyclononene L (0.05 mmol). LC-MS: 0.94 min, MH+=665.26.

### Example 13

### (rac.)-(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]non-6-ene-3-carboxylic acid methyl ester

According to general procedure B, from bicyclononene K (0.05 mmol). LC-MS: 0.94 min, MH+=665.26.

### Example 14

### (rac.)-(1R*,3R*,5S*)-3-{4-[3-(2-Chloro-3,6-difluorophenoxy)propyl]-phenyl}-7-methoxymethyl-9-azabicyclo[3.3.1]non-2-ene-2-carboxylic acid cyclopropyl-(3-methoxy-2-methylbenzyl)amide

A mixture of compound M1 (37.6 mg, 0.05 mmol), MeI (4.05 μL, 0.065 mmol), NaH (55%, 2.4 mg, 0.055 mmol), and 15-crown-5 (9.9 μL, 0.05 mmol) in THF (1 mL) was stirred at rt overnight. The reaction mixture was poured over diatomace earth (Isolute Sorbent Technology, Johnson, C. R., et al., Tetrahedron, 1998, 54, 4097; 0.5 g), and was treated with water (0.6 mL). The diatomace earth-suspension was left for 5 min, and was washed with CH₂Cl₂ (2×). The org. extracts were evaporated under reduced pressure. The residue was used without further purification in general procedure B. LC-MS: 1.02 min; ES+: 665.27.

### Example 15

### (rac.)-(1R*,3R*,5S*)-3-{4-[3-(2-Chloro-3,6-difluorophenoxy)propyl]phenyl}-7-cyclopropoxymethyl-9-aza-bicyclo[3.3.1]non-2-ene-2-carboxylic acid cyclopropyl-(3-methoxy-2-methylbenzyl)amide

A mixture of compound M1 (37.6 mg, 0.05 mmol), bromomethylcyclopropane (6.21 μL, 0.065 mmol), NaH (55%, 2.4 mg, 0.055 mmol), and 15-crown-5 (9.9 μL, 0.05 mmol) in THF (1 mL) was stirred at rt overnight. The reaction mixture was poured over diatomace earth (Isolute Sorbent Technology, Johnson, C. R., et al., Tetrahedron, 1998, 54, 4097; 0.5 g), and was treated with water (0.6 mL). The diatomace earth-suspension was left for 5 min, and was washed with CH₂Cl₂ (2×). The org. extracts were evaporated under reduced pressure. The residue was used without further purification in general procedure B. LC-MS: 1.01 min; ES+: 705.28.

### Example 16

### (rac.)-(1R*,3R*,5S*)-7-Aminomethyl-3-{4-[3-(2-chloro-3,6-difluorophenoxy)propyl]phenyl}-9-azabicyclo[3.3.1]non-2-ene-2-carboxylic acid cyclopropyl-(3-methoxy-2-methylbenzyl)amide

From compound M3, according to general procedure B. LC-MS: 0.82 min; ES+: 650.25.

### Example 17

### (rac.)-(1R*,3R*,5S*)-7-(Acetylaminomethyl)-3-{4-[3-(2-chloro-3,6-difluorophenoxy)propyl]phenyl}-9-azabicyclo[3.3.1]non-2-ene-2-carboxylic acid cyclopropyl-(3-methoxy-2-methylbenzyl)amide

A mixture of compound M3 (67.5 mg, 0.09 mmol), Amberlyst IRA 67 (100 mg), and acetyl chloride (19.2 mL, 0.27 mmol) in CH₂Cl₂ (2 mL) was stirred at rt overnight. Water was added, and the mixture was stirred for 1 h. The mixture was filtered, and the filtrate was evaporated under reduced pressure. The residue was proceeded further according to general procedure B. LC-MS: 0.92 min; ES+: 692.27.

### Example 18

### (rac.)-(1R*,3R*,5S*)-3-{4-[3-(2-Chloro-3,6-difluorophenoxy)propyl]-phenyl}-7-dimethylaminomethyl-9-azabicyclo[3.3.1]non-2-ene-2-carboxylic acid cyclopropyl-(3-methoxy-2-methylbenzyl)amide

A mixture of compound M3 (135 mg, 0.18 mmol), formaldehyde (36.5% in water, 27.6 μL, 0.36 mmol), and NaBH(OAc)₃ (53.4 mg, 0.25 mmol) in CH₂Cl₂ was stirred at rt overnight. Aq. 1M NaOH (0.2 mL) was added. The mixture was poured over diatomace earth (Isolute Sorbent Technology, Johnson, C. R., et al., Tetrahedron, 1998, 54, 4097; 0.5 g), and was treated with aq. 1M NaOH (0.7 mL). The diatomace earth-suspension was left for 5 min, and was washed with CH₂Cl₂ (3×). The org. extracts were evaporated under reduced pressure. The residue was used without further purification in general procedure B. LC-MS: 0.83 min; ES+: 678.30.

### Example 19

### (rac.)-(1R*,3R*,5S*)-3-{4-[3-(2-Chloro-3,6-difluorophenoxy)propyl]phenyl}-7-hydroxymethyl-9-azabicyclo[3.3.1]non-2-ene-2-carboxylic acid cyclopropyl-(3-methoxy-2-methylbenzyl)amide

From compound M1, according to general procedure B. LC-MS: 0.89 min; ES+: 650.27.

Inhibition of Human Recombinant Renin by the Compounds of the Invention

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:
human recombinant renin (0.16 ng/mL)•synthetic human angiotensin(1-14) (0.5 μM)
hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang 1-BSA). 75 μL of Ang I-antibodies in essaybuffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at room temperature. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀). The IC₅₀-values of all compounds tested are below 100 nM. However selected compounds exhibit a very good bioavailibility and are metabolically more stable than prior art compounds.

1. Compounds of the general formula I 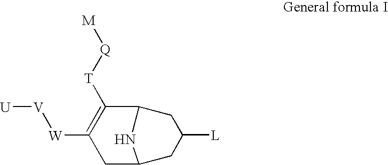
wherein
W is a six-membered, non benzofused, phenyl or heteroaryl ring, substituted by V in meta or para position;
V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O—; or —O—C(CH₂CH₂)—CH₂—O—;
A and B independently represent —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; or —COO—;
Q represents lower alkylene; lower alkenylene;
M represents hydrogen; cycloalkyl; aryl; heterocyclyl; heteroaryl; aryl-O(CH₂)ᵥR⁵—; heteroaryl-O(CH₂)ᵥR⁵—; aryl-O(CH₂)₂O(CH₂)_{w}R⁵—; heteroaryl-(CH₂)₂O(CH₂)_{w}R⁵—;
L represents —H; —CH₂OR³; —CH₂NR²R³; —CH₂NR²COR³; —CH₂NR²SO₂R³; —CO₂R³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkinyl; cycloalkyl; aryl; cycloalkyl -lower alkyl;
R² and R²′ independently represent hydrogen; lower alkyl; lower alkenyl; cycloalkyl; cycloalkyl -lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted or mono-, di- or trisubstituted with hydroxy, —OCOR², —COOR², lower alkoxy, cyano, —CONR²R²′, —CO-morpholin-4-yl, —CO-((4-loweralkyl)piperazin-1-yl), —NH(NH)NH₂, —NR⁴R⁴′ or lower alkyl, with the proviso that a carbon atom is attached at the most to one heteroatom in case this carbon atom is sp3-hybridized;
R⁴ and R⁴′ independently represents hydrogen; lower alkyl; cycloalkyl; cycloalkyl-lower alkyl; hydroxy-lower alkyl; —COOR²; —CONH₂;
R⁵ represents —OH, —OCOR², —COOR², —NR²R²′, —OCONR²R²′, —NCONR²R²′, cyano, —CONR²R²′, SO₃H, —SONR²R²′, —CO-morpholin-4-yl, —CO-((4-loweralkyl)piperazin-1-yl), —NH(NH)NH₂, —NR⁴R⁴′, with the proviso that a carbon atom is attached at the most to one heteroatom in case this carbon atom is sp3-hybridized;
p is the integer 1, 2, 3 or 4;
r is the integer 3, 4, 5, or 6;
s is the integer 2, 3, 4, or 5;
t is the integer 1, 2, 3, or 4;
u is the integer 1, 2, or 3;
v is the integer 2, 3, or 4;
w is the integer 1 or 2;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

2. Compounds of general formula I according to claim 1 wherein W, V, U, and L are as defined in general formula I and
T represents —CONR¹—;
Q represents methylene;
M represents aryl, heteroaryl;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

3. Compounds of general formula I according to claim 1 wherein W, U, L, T, Q, and M are as defined in general formula I and
V represents —CH₂CH₂O—; —CH₂CH₂CH₂O—; —OCH₂CH₂O—;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

4. Compounds of general formula I according to claim 1 wherein V, U, T, Q, M, and L are as defined in general formula I and
W represents a 1,4-disubstituted phenyl group;
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

5. Compounds of general formula I according to claim 1 wherein W, V, Q, T, M, and L are as defined in general formula I and
U is a mono-, di-, or trisubstituted phenyl or heteroaryl, whereby the substituents are halogen, lower alkyl, lower alkoxy, CF₃
and optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

6. The compound according to claim 1 which is
(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-hydroxyethyl)amide], in free or pharmaceutically acceptable salt form.

7. Pharmaceutical compositions comprising at least one compound according to claim 1 and a pharmaceutically acceptable carrier.

8. A method for the treatment or prophylaxis of diseases which are related to the renin-angiotensin system comprising hypertension, congestive heart failure, pulmonary hypertension, cardiac insufficiency, renal insufficiency, renal or myocardial ischemia, atherosclerosis, renal failure, erectile dysfunction, glomerulonephritis, renal colic, glaucoma, diabetic complications, complications after vascular or cardiac surgery, restenosis, complications of treatment with immunosuppressive agents after organ transplantation, and other diseases which are related to the RAS, which method comprises administering an effective amount of at least one compound according to claim 1 to a human being or animal in need of such treatment or prophylaxis.

9. (canceled)

10. The method of claim 8 further comprising administration of a pharmacologically active compounds selected from ACE inhibitors, angiotensin II receptor antagonists, endothelin receptor antagonists, vasodilators, calcium antagonists, potassium activators, diuretics, sympatholitics, beta-adrenergic antagonists, alpha-adrenergic antagonists, and neutral endopeptidase inhibitors.

11. A compound according to claim 1 which is:
(1R*,5S*,7R*)-3-{4-[3-(2-chloro-3,6-difluorophenoxy)propyl]phenyl}-9-azabicyclo[3.3.1]non-2-ene-2,7-dicarboxylic acid 7-benzylamide 2-[cyclopropyl-(3-methoxy-2-methylbenzyl)amide, in free or pharmaceutically acceptable salt form.

12. A compound according to claim 1 which is:
(1R*,5S*,7R*)-3-{4-[3-(2-chloro-3,6-difluorophenoxy)propyl]phenyl}-7-(4-methylpiperazine-1-carbonyl)-9-azabicyclo[3.3.1]non-2-ene-2-carboxylic acid cyclopropyl-(3-methoxy-2-methylbenzyl)amide, in free or pharmaceutically acceptable salt form.

13. A compound according to claim 1 which is:
(1R*,5S*,7R*)-3-{4-[3-(2-chloro-3,6-difluorophenoxy)propyl]phenyl}-9-azabicyclo[3.3.1]non-2-ene-2,7-dicarboxylic acid 7-cyclopropylamide 2-[cyclopropyl-(3-methoxy-2-methylbenzyl)amide], in free or pharmaceutically acceptable salt form.

14. A compound according to claim 1 which is:
(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-diethylamide, in free or pharmaceutically acceptable salt form.

15. A compound according to claim 1 which is:
(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-[(2-piperidin-1-ylethyl)amide], in free or pharmaceutically acceptable salt form.

16. A compound according to claim 1 which is:
(1R*,5S*,7R*)-3-{4-[3-(2-chloro-3,6-difluorophenoxy)propyl]phenyl}-7-(4-hydroxypiperidine-1-carbonyl)-9-azabicyclo[3.3.1]non-2-ene-2-carboxylic acid cyclopropyl-(3-methoxy-2-methylbenzyl)amide in free or pharmaceutically acceptable salt form.

17. A compound according to claim 1 which is:
(1R*,5S*,7R*)-3-{4-[3-(2-chloro-3,6-difluorophenoxy)propyl]phenyl}-7-(2-hydroxymethylpyrrolidine-1-carbonyl)-9-azabicyclo[3.3.1]non-2-ene-2-carboxylic acid cyclopropyl-(3-methoxy-2-methylbenzyl)amide, in free or pharmaceutically acceptable salt form.

18. A compound according to claim 1 which is:
(1R*,5S*,7R*)-3-{4-[3-(2-chloro-3,6-difluorophenoxy)propyl]phenyl}-9-aza-bicyclo[3.3.1]non-2-ene-2,7-dicarboxylic acid 2-[cyclopropyl-(3-methoxy-2-methylbenzyl)amide]7-[(2-methoxyethyl)amide], in free or pharmaceutically acceptable salt form.

19. A compound according to claim 1 which is:
(1R*,5S*,7R*)-3-{4-[3-(2-chloro-3,6-difluorophenoxy)propyl]phenyl}-9-azabicyclo[3.3.1]non-2-ene-2,7-dicarboxylic acid 2-[cyclopropyl-(3-methoxy-2-methylbenzyl)amide]7-methylamide, in free or pharmaceutically acceptable salt form.

20. A compound according to claim 1 which is:
({-(1R*,3R*,5S*)-7-{4-[3-(2-chloro-3,6-difluorophenoxy)propyl]phenyl}-6-[cyclopropyl-(3-methoxy-2-methylbenzyl)carbamoyl]-9-azabicyclo[3.3.1]non-6-ene-3-carbonyl}amino)acetic acid methyl ester, in free or pharmaceutically acceptable salt form.

21. A compound according to claim 1 which is:
(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]non-6-ene-3-carboxylic acid, in free or pharmaceutically acceptable salt form.

22. A compound according to claim 1 which is:
(1R*,3R*,5S*)-7-{4-[3-(2-chloro-3,6-difluorophenoxy)propyl]phenyl}-6-[cyclopropyl-(3-methoxy-2-methylbenzyl)-carbamoyl]-9-azabicyclo[3.3.1]non-6-ene-3-carboxylic acid methyl ester, in free or pharmaceutically acceptable salt form.

23. A compound according to claim 1 which is:
(1R*,3R*,5S*)-3-{4-[3-(2-chloro-3,6-difluorophenoxy)propyl]phenyl}-7-methoxymethyl-9-azabicyclo[3.3.1]non-2-ene-2-carboxylic acid cyclopropyl-(3-methoxy-2-methylbenzyl)amide, in free or pharmaceutically acceptable salt form.

24. A compound according to claim 1 which is:
(1R*,3R*,5S*)-3-{4-[3-(2-chloro-3,6-difluorophenoxy)propyl]phenyl}-7-cyclopropoxymethyl-9-azabicyclo[3.3.1]non-2-ene-2-carboxylic acid cyclopropyl-(3-methoxy-2-methylbenzyl)amide, in free or pharmaceutically acceptable salt form.

25. A compound according to claim 1 which is:
(1R*,3R*,5S*)-7-aminomethyl-3-{4-[3-(2-chloro-3,6-difluorophenoxy)-propyl]phenyl}-9-azabicyclo[3.3.1]non-2-ene-2-carboxylic acid cyclopropyl-(3-methoxy-2-methylbenzyl)amide, in free or pharmaceutically acceptable salt form.

26. A compound according to claim 1 which is:
(1R*,3R*,5S*)-7-(acetylaminomethyl)-3-{4-[3-(2-chloro-3,6-difluoro-phenoxy)propyl]phenyl}-9-azabicyclo[3.3.1]non-2-ene-2-carboxylic acid cyclo-propyl-(3-methoxy-2-methylbenzyl)amide, in free or pharmaceutically acceptable salt form.

27. A compound according to claim 1 which is:
(1R*,3R*,5S*)-3-{4-[3-(2-chloro-3,6-difluorophenoxy)propyl]phenyl}-7-hydroxymethyl-9-azabicyclo[3.3.1]non-2-ene-2-carboxylic acid cyclopropyl-(3-methoxy-2-methylbenzyl)amide, in free or pharmaceutically acceptable salt form.

28. A compound according to claim 1 which is:
(1R*,3R*,5S*)-3-{4-[3-(2-chloro-3,6-difluorophenoxy)propyl]phenyl}-7-dimethylaminomethyl-9-azabicyclo[3.3.1]non-2-ene-2-carboxylic acid cyclo-propyl-(3-methoxy-2-methylbenzyl)amide, in free or pharmaceutically acceptable salt form.

29. A method of making a compound of general formula I according to claim 1 comprising deprotecting a compound of formula M: 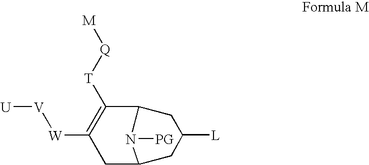
wherein L, M, Q, T, U, V, and W are as defined for claim 1 and PG is a protecting group, and isolating the compound of general formula I thus obtained.
